(19) United States
(12) Patent Application Publication
(10) Pub. No.: US 2026/0102190 A1
(43) Pub. Date: Apr. 16, 2026

(54) TOE REALIGNMENT SYSTEM AND RELATED DEVICES AND METHODS

(71) Applicant: Veritus Surgical Consulting Enterprises LLC, San Antonio, TX (US)

(72) Inventors: Walter Strash, Canyon Lake, TX (US); Isabella Strash, Canyon Lake, TX (US); Abelardo Osuna, Laredo, TX (US); Thomas Zink, San Antonio, TX (US)

(21) Appl. No.: 19/355,235

(22) Filed: Oct. 10, 2025

Related U.S. Application Data
(60) Provisional application No. 63/705,775, filed on Oct. 10, 2024.

(51) Int. Cl. A61B 17/80; A61B 17/14

(52) U.S. Cl. CPC A61B 17/8095; A61B 17/14

(57) ABSTRACT

The various embodiments disclosed herein relate to a toe realignment system and devices and methods that aid in the accurate realignment of toes. For example, a toe realignment system may include a saw blade to cut a single cut into a proximal phalanx of a toe, a wedge having a perimeter having lateral and proximal ends and an interior portion. The wedge may have a lateral thickness and a medial thickness, with the lateral thickness larger than the medial thickness. There may also be a connection member configured to secure the wedge to the proximal phalanx.

### CROSS-REFERENCE TO RELATED APPLICATION

This application claims the benefit under 35 U.S.C. § 119(e) to U.S. Provisional Application 62/705,775, filed Oct. 10, 2024 and entitled “Toe Realignment System and Related Devices and Methods,” which is hereby incorporated herein by reference in its entirety.

### FIELD

The various embodiments herein relate to surgical systems, devices, and methods of realigning a toe suffering from hallux valgus. The systems and methods disclosed herein result in a realignment of the toe with fewer required cuts into the bone and a more reliable and accurate realignment angle.

### BACKGROUND

The “Akin” osteotomy procedure is a well-known method for correcting hallux valgus, the condition also referred to as a “bunion” and depicted in FIG. 1 in a foot 10 having toes 12, 14, 16, 18, 20. One version of the known Akin procedure is depicted in FIGS. 2A-2C. First, cuts are made in the proximal phalanx 22 of the big toe 20 to form a bone wedge 40, as shown in FIG. 2A. That wedge 40 is then removed, as shown in FIG. 2B. Finally, the proximal phalanx 22 is then repositioned at the gap created by the removal of the bone wedge 40 to realign the proximal phalanx 22 and thus the entire big toe 20 as shown in FIG. 2C.

One disadvantage of the Akin procedure is that two separate bone cuts are necessary to create and remove the angled wedge which realigns the toe to the desired angle. If these cuts are not parallel to one another the proximal phalanx may be placed in an incorrect dorsiflexed or plantarflexed position, creating further complications to the patient. A saw cut through the lateral cortex when making two cuts will cause rotation of the bone in the frontal plane creating malposition of the toe. The patient's toe nail may not be facing straight up in this malposition.

There is a need in the art for an improved method and device for correcting hallux valgus.

### BRIEF SUMMARY

Discussed herein are various systems, methods, and devices for correcting a toe suffering from hallux valgus.

In Example 1, a system for realigning a tow includes a saw blade configured to cut a single cut into a proximal phalanx of a toe, thereby creating proximal and distal portions of the proximal phalanx connected by a hinge portion. The system includes a wedge having a perimeter having lateral and proximal ends and an interior portion. The wedge has a lateral thickness and a medial thickness, and the lateral thickness is larger than the medial thickness. The system further includes a connection member configured to secure the wedge to the proximal phalanx.

Example 2 relates to the system according to Example 1, wherein the saw blade is configured to cut the single cut in a wedge shape into the proximal phalanx of the toe.

Example 3 relates to the system according to Example 1, wherein the single cut has a lateral cut thickness, and wherein the lateral thickness of the wedge is greater than the lateral cut thickness of the wedge-shaped cut.

Example 3 relates to the system according to Example 3, wherein the lateral thickness of the wedge is greater than the lateral cut thickness of the wedge-shaped cut by an amount that corresponds to a predetermined angle change thereby straightening the toe.

Example 5 relates to the system according to Example 1, wherein the wedge comprises a polyether ether ketone (PEEK) material.

Example 6 relates to the system according to Example 1, wherein the connection member comprises a suture.

Example 7 relates to the system according to Example 1, wherein the connection member comprises a staple.

Example 8 relates to the system according to Example 7, wherein the staple is integrally formed with the wedge.

Example 9 relates to the system according to Example 8, wherein the staple comprises legs configured to couple to the proximal phalanx by locating the legs within staple holes drilled into each of the proximal and distal portions of the proximal phalanx.

In Example 10, a method of realigning a toe includes the steps of creating a hinge by cutting a single cut into the lateral side of the proximal phalanx of the toe, straightening the toe by rotating a distal portion of the proximal phalanx about the hinge, thereby creating a wedge opening defined by the space between the distal portion of the proximal phalanx, a proximal portion of the proximal phalanx, and the hinge, inserting a wedge into the wedge opening thereby providing support to the proximal phalanx, and securing the wedge to the proximal phalanx.

Example 11 relates to the method of Example 10, wherein the securing the wedge step is performed by suturing the wedge to the proximal phalanx.

Example 12 relates to the method of Example 10, wherein the securing the wedge step is performed by stapling the wedge to the proximal phalanx.

Example 13 relates to the method of Example 12, wherein the stapling the wedge step is performed by using a staple attached to the wedge, the staple having legs configured to couple to the proximal phalanx by locating the legs within staple holes drilled into each of the proximal and distal portions of the proximal phalanx.

Example 14 relates to the method of Example 13, wherein the staple is integrally formed with the wedge.

Example 15 relates to the method of Example 10, wherein the creating a hinge step is performed using a reciprocating saw.

Example 16 relates to the method of Example 15, wherein the reciprocating saw has a reciprocating saw blade.

Example 17 relates to the method of Example 16, wherein the reciprocating saw blade is configured to take a wedge shaped cut into the proximal phalanx of the toe.

In Example 18, a system for realigning a toe includes a wedge having a perimeter, lateral and medial ends; and an interior portion. The wedge has a lateral thickness associated with the lateral end and a medial thickness associated with the medial end, and wherein the lateral thickness is larger than the medial thickness. The system includes a connection member to secure the wedge to a proximal phalanx of the toe. The proximal phalanx includes a proximal portion, a distal portion separated from the proximal portion by a cut substantially through the proximal phalanx, and a hinge portion connecting the distal portion to the proximal portion.

Example 19 relates to Example 18, wherein the connection member is a staple formed integrally with the wedge, and wherein the staple comprises a pair of prongs that correspond to a pair holes in the proximal phalanx.

Example 20 relates to Example 19, wherein one of the pair of holes is disposed in the proximal portion of the proximal phalanx, and the other of the pair of holes in disposed in the distal portion of the proximal phalanx.

While multiple embodiments are disclosed, still other embodiments will become apparent to those skilled in the art from the following detailed description, which shows and describes various illustrative implementations. As will be realized, the various embodiments herein are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a top view of a prior art fix for a bunion treatment.

FIG. 2A is a closeup top view showing the cutting method of the prior art bunion treatment of FIG. 1.

FIG. 2B is a closeup top view showing a wedge being removed using the cutting method of the prior art bunion treatment of FIG. 2A.

FIG. 2C is a closeup top view showing a re-oriented toe using the cutting method of the prior art bunion treatment of FIG. 2A.

FIG. 2D is a top view of a foot prior to a bunion treatment, according to one embodiment.

FIG. 2E is a top view of the foot of FIG. 2D after a bunion treatment with a wedge, according to one embodiment.

FIG. 3A is a top view of a toe showing the initial cut of a bunion treatment according to one embodiment.

FIG. 3B is a top view of a toe showing the cut and rotation of a portion of the toe of a bunion treatment according to one embodiment.

FIG. 3C is a top view of a toe showing the final set using a wedge of a bunion treatment according to one embodiment.

FIG. 3D is a top view of a toe in relation to the entire foot showing the final set using a wedge of a bunion treatment according to one embodiment.

FIG. 4 is a top view a toe showing an initial cut and staple holes according to one embodiment.

FIG. 5 is a side perspective view of a saw blade for use in a method for treating a bunion according to one embodiment.

FIG. 6 is a schematic view of a portion of a toe showing the final set using a wedge of a bunion treatment according to one embodiment.

FIG. 7A is a side perspective view of a wedge according to one embodiment.

FIG. 7B is side perspective view of a wedge according to another embodiment.

FIG. 7C is a side perspective view of a wedge according to yet another embodiment.

FIG. 7D is a side perspective view of a drill jig according to one embodiment.

### DETAILED DESCRIPTION

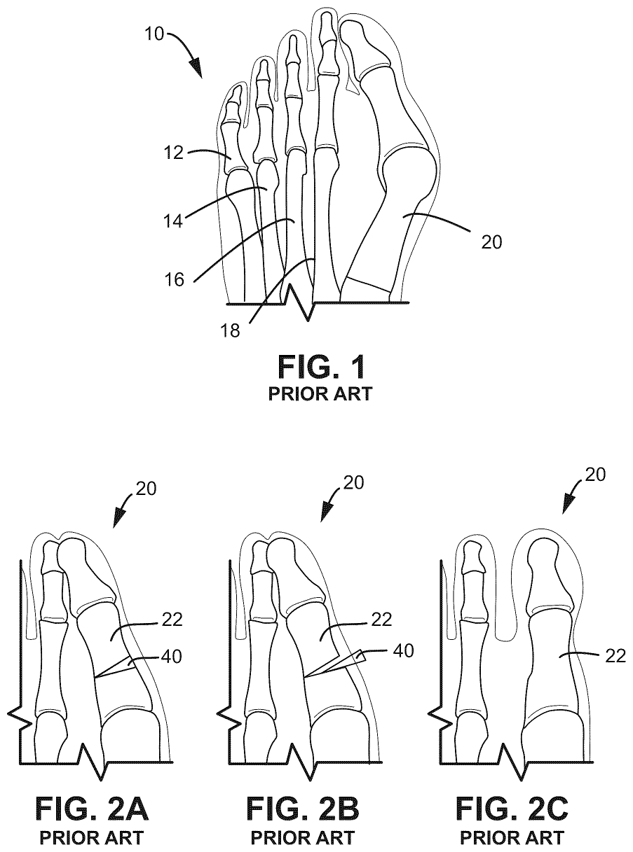

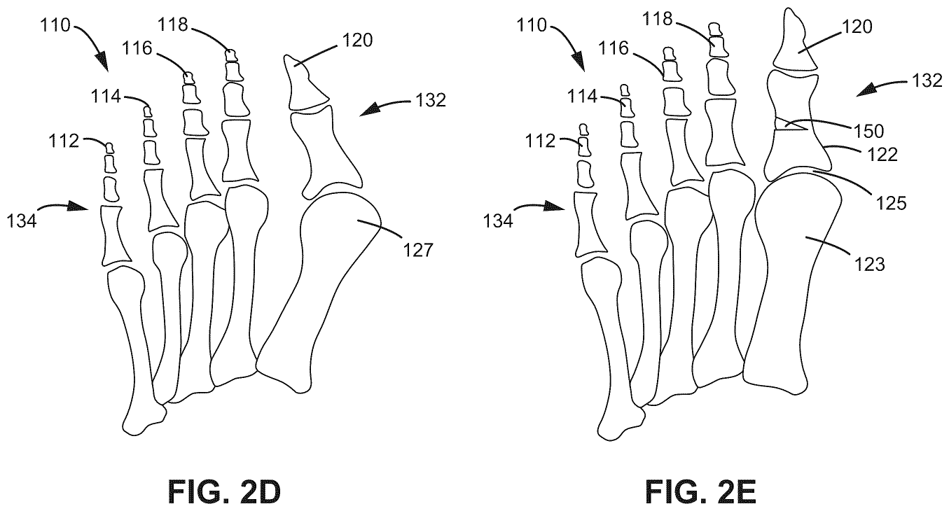

The various hallux valgus treatment device and method embodiments disclosed or contemplated herein include a method and a system of realigning a toe by adjusting the alignment of the toe using a wedge, broadly shown in FIGS. 2D-2E. As used herein, the term “wedge” is intended to mean an implant that has a wedge or pie-like shape. In one embodiment, the wedge 150 is inserted into the proximal phalanx as shown in FIG. 2E, which urges the distal portion of the proximal phalanx 122 toward the medial side 132 of the foot, pulling the distal portion of the proximal phalanx along with the distal phalanx of the big toe 120 which is connected at a distal end of the proximal phalanx away from the neighboring toe 118, thereby treating the hallux valgus or bunion 127 previously exhibited in the target toe 120 as shown in FIG. 2D. The wedge 150 may be secured to the bone such that it remains stationary until the bone heals around and/or through the wedge.

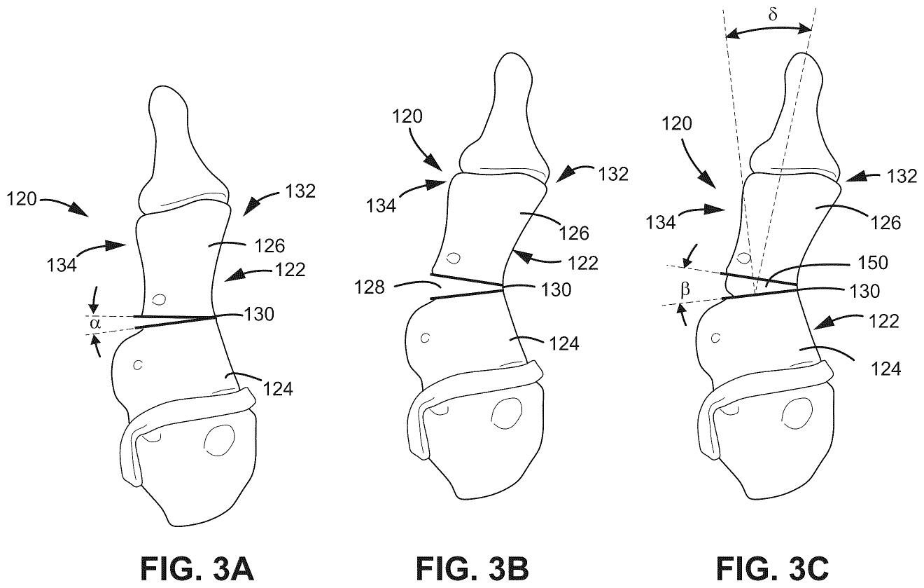

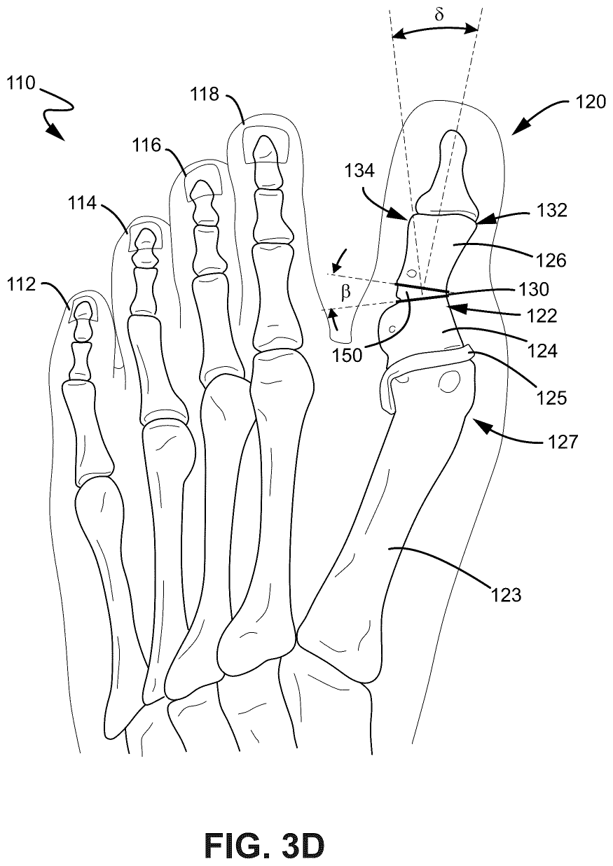

As best shown in FIGS. 3A-3C, the process for realigning a toe (e.g., rotating the distal portion of a big toe 120 on a patient's left foot having a medial side 132 and a lateral side 134) with hallux valgus using a wedge as disclosed or contemplated herein includes cutting the bone (e.g., the proximal phalanx 122) to create a gap 128 (as best shown in FIG. 3B), realigning the distal end 126 of the proximal phalanx 122 of the big toe 120 by some degree d (as best shown in FIG. 3C), inserting a wedge 150 into the now enlarged gap 128, and securing the wedge to the bone (described and shown in further detail below). Securing the wedge to the bone restricts movement, fixes the bone realignment in place, and allows the toe to heal. In certain embodiments, the cut may be a wedge-shaped cut 128 having an angle a, the wedge 150 may have an angle b, and the overall change or realignment angle d of the direction of the toe 120 may be calculated using the exemplary, non-limiting formula below:

$$d = b - a$$

Where:
d is the total angle of realignment of the toe;
b is the angle of the wedge itself; and
a is the angle of the wedge-shaped cut, if any.

It should be noted that in an embodiment including a straight cut (i.e., not a wedge-shaped cut but rather a single, straight across cut) where a=0, then the overall change angle d will equal the wedge angle b itself. Each of these steps and the associated elements to accomplish the steps are described in detail below.

As best shown in FIG. 3D, the resulting angle change d of the toe 120 with respect to the rest of the foot 110 and the adjacent toe 118 is shown. The foot 110 may include toes 112, 114, 116, 118, and 120. Big toe 120 may include proximal phalanx 122 disposed distally from a metatarsal 123 and joined at a metatarsal phalanx joint (MTPJ) 125. The proximal phalanx 122 is shown with a wedge 150 having an angle b inserted into the gap created by the cut into that bone. In such an embodiment, the wedge 150 realigns the distal portion 126 of the proximal phalanx 122 with respect to a proximal portion 124 by an angle d. In the non-limiting embodiment shown, this realignment urges the toe toward the medial side 132 and away from the lateral side 134 which increases the distance between the big toe 120 from the adjacent toe 118 reducing the effect of the bunion 127.

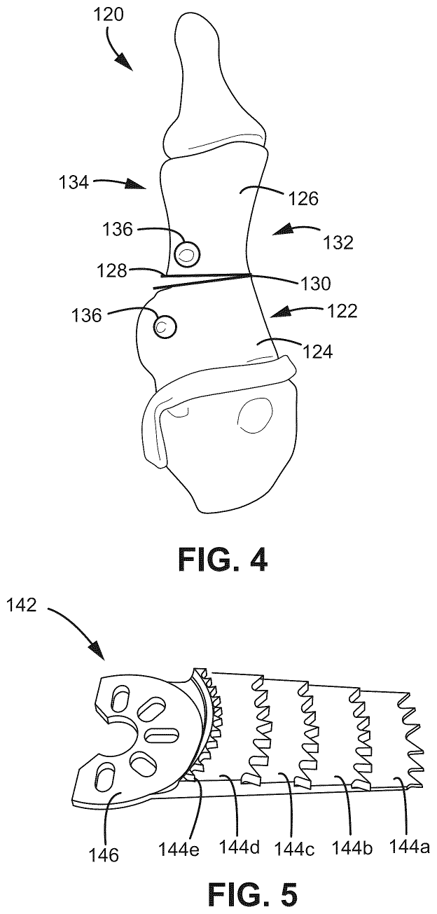

As best shown in FIG. 4, the first step is to take a cut out of the bone to provide a space or gap 128 to allow the practitioner to manipulate the orientation of the distal portion 126 of the proximal phalanx 122 with respect to the proximal portion 124. The cut that is taken may be any size, shape, and configuration in order to provide a space 128 to allow manipulation of the distal portion 126 with respect to the proximal portion 124 of the proximal phalanx 122 and within which to place a wedge 150, as is described in further detail below.

In some embodiments, the cut may be a wedge-shaped cut from a wedge-shaped saw blade 142 as shown in FIG. 5. In some embodiments, the wedge-shaped saw blade 142 may include a number of reciprocating saw blades 144a-e stacked in a gradually offset formation extending from a saw connection section 146 (e.g., a typical reciprocating saw connection such as shown in FIG. 5, although it should be known that other saw connections may be used without deviating from the scope of the disclosure). This stacked offset formation allows for a narrower cut toward the medial side 132 of the bone and a wider cut on the lateral side 134 of the bone (as oriented and shown in FIG. 4). In other embodiments, the wedge-shaped cut may be made by a traditional blade in two parts to create the wedge-shaped cutout 128 desired. In still other embodiments, a single, straight cut may be made across the bone.

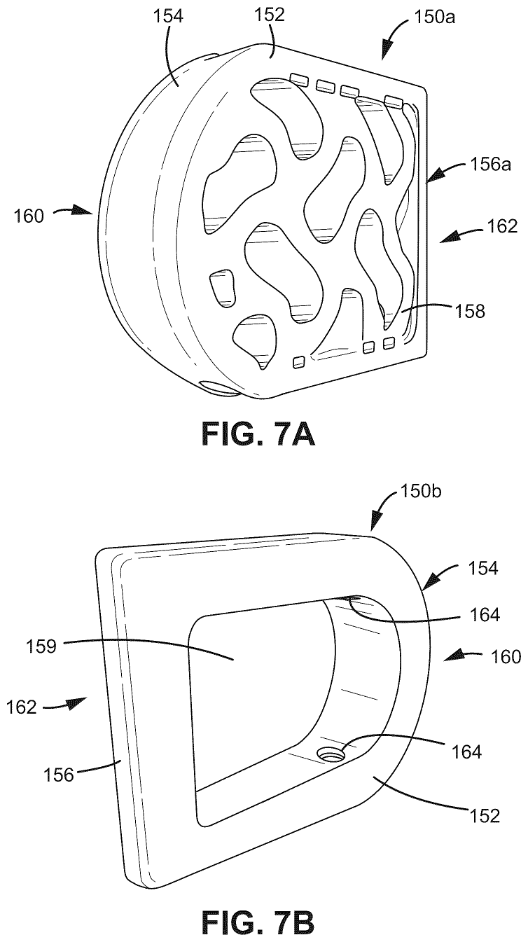

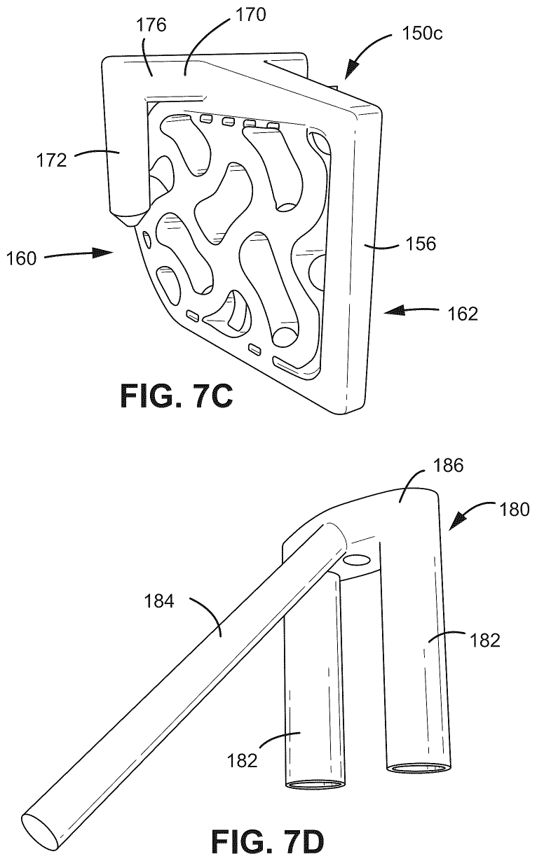

Returning to FIG. 4, in some embodiments, one or more holes 136 may be drilled into either or both of the proximal and distal portions 124, 126. The holes 124, 176 in such embodiments may be configured to attach legs 172 of a staple 170 (see FIG. 7C described in detail, below) that may be coupled to the wedge 150 to ensure secure attachment of the wedge 150 to the proximal phalanx 122.

Continuing with FIG. 4, the cut results in a proximal phalanx 122 that is split into a proximal portion 124 and a distal portion 126 (as noted above), in some embodiments still connected by a hinge portion 130. In some embodiments, the cut is not made all the way through the bone, but rather leaves a bit of the medial side 132 of the bone to use as a hinge point 130 about which the distal portion 126 of the proximal phalanx 122 may be rotated away from the adjacent toe (e.g., second toe 18 as shown in FIG. 1). In other embodiments, the cut may be made all the way through and the distal and proximal portions 124, 126 of the proximal phalanx 122 reconnected along with the wedge 150 in any fashion known in the art. In still other embodiments, a single thickness blade (not shown) may be used to simply make a single, constant-thickness cut most of the way across the proximal phalanx 122, while leaving a hinge portion 130 on the medial side 132 of the proximal phalanx 122.

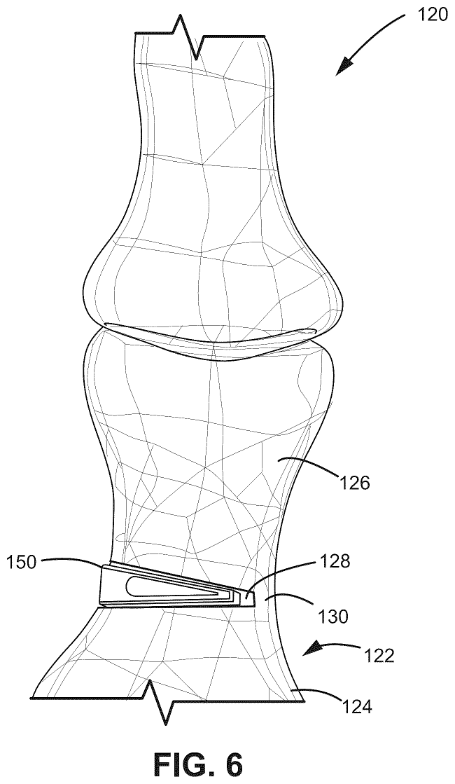

In some embodiments (as shown previously in FIGS. 3A-3C), once the proximal phalanx has been cut as described above, the distal portion 126 of the proximal phalanx 122 may rotated at some angle d with respect to the proximal portion 124 of the proximal phalanx 122, thereby increasing the gap 128 and preparing for the wedge 150 to be inserted into and secured within. As best shown in FIG. 6, the wedge 150 can be inserted into or otherwise positioned within the gap 128 such that the wedge 128 is disposed within the gap 128 between the proximal and distal portion 124, 126 of the proximal phalanx 122 as the distal portion 126 hinges about the hinge point 130. As such, the wedge 128 changes and establishes the angle that the distal portion 126 of the proximal phalanx 122 extends from the proximal portion 124 (in comparison to when there is no wedge 128 disposed in the gap 128) and by extension the angle of the tip of the toe 120 and its orientation with respect to an adjacent toe (e.g., second toe 18 as shown in FIG. 1).

As best shown in FIGS. 7A-7C, the wedge 150 may be any size, shape, and configuration in order to be securely disposed between the distal and proximal portions 126, 124 and thereby hold the distal portion 126 in the desired and/or predetermined orientation in relation to the proximal portion 124 during the healing process. In some embodiments, the wedge 150 may be a D-shaped wedge 150 having a perimeter (or “body”) 152, an outside surface 154, and interior surface 156, interior wall structure 158, and a lateral end 160 and medial end 162. More specifically, as discussed in further detail below, FIG. 7A depicts a first wedge embodiment 150a with an internal porous structure 158, FIG. 7B depicts a second wedge embodiment 150b with an opening 159 defined through the body 152 (rather than a porous structure), and FIG. 7C depicts a third wedge embodiment 150c with an internal porous structure and a staple 170 associated with the wedge 150c.

For each of the wedge implementations 150a-150c, the thickness of the wedge 150 may increase from the narrowest part at the medial end 162 and grow thicker toward the thickest part at the lateral end 160 of the body 152. In such embodiments, the lateral end 160 of the wedge 150 may measure approximately 2 mm in thickness, while the medial end 162 of the wedge 150 may measure approximately 1 mm in thickness. In some embodiments, the outside surface 154 (i.e., the surface that forms the D-structure of the wedge) at the lateral end 160 of the body 152 is formed to substantially match the outer surface of the proximal phalanx 122 at the point of insertion.

Turning now to the embodiment of FIG. 7A, the interior of the wedge 150a has a porous interior wall structure 158 to increase the strength of the wedge 150. Further, in certain embodiments, the network of pores in the porous structure 158 can encourage bone ingrowth within the pores. Although it should be known by those of skill in the art that this interior porous structure 158 may be omitted as shown in the wedge 150b in FIG. 7B.

In some embodiments, the wedge 150b (or any wedge embodiment herein) may include through holes 164. These through holes 164 may be threaded or unthreaded through holes 164. The through holes 164 may provide the practitioner a hole for securing the wedge 150 to the bone structure (e.g., using a screw or the like to fasten the wedge and bone structure).

In some embodiments such as shown in FIG. 7C, the wedge 150c can have a staple structure 170 attached to or integrated into the wedge 150c that may be used to secure the wedge 150c into place in the target bone that is being treated. It should be understood that the staple 170 may be any size, shape, and configuration in order to attach the wedge 150c to the surrounding bone structure (e.g., the proximal and distal portions 124, 126 of the proximal phalanx 122) in order to keep them stable and secure during the healing process. According to the specific embodiment of FIG. 7C, the wedge 150c has a staple 170 that includes a base 176 extending laterally across a top portion of the wedge 150c. At either end of the base 176 is a pair of prongs 172 extending downwardly at some angle as shown.

In such an embodiment, the prongs (or “tines”) 172 of the staple 170 may be sized, shaped, and located to fit into the holes 136 drilled into the proximal phalanx 122 as shown previously in FIG. 4. The prongs 172 may be sized such that they are press fit into the holes 136 to hold the staple 170 securely in place while the bone heals around the wedge 150. In other embodiments, a separate fastener (e.g., a screw or the like) may be passed through the wedge 150 and into the bone. In still other embodiments, bone graft may be placed within the space 159 of the wedge 150b or between the lattice structure 158 of wedge 150a to aid in the bone healing process and reduce the time necessary to secure the wedge in place (i.e., the bone may heal faster thus reducing the time to artificially secure the wedge in place). Alternatively, the staple 170 structure can be disposed anywhere along the body 152 of the wedge 150c such that the prongs 172 can be inserted into corresponding holes formed in the distal and proximal portions 124, 126 of the proximal phalanx 122 as discussed above.

As best shown in FIG. 7D, some embodiments include a drill jig 180 to aid the practitioner in locating and drilling holes to accept the prongs 172 of the staple 170 as shown in FIG. 7C. In such embodiments, the holes 136 may be drilled into the proximal phalanx 122 using the drill jig 180. The drill jig 180 may be any size, shape, and configuration to aid a practitioner to drill the holes 136 in the correct location for the staple 170. The drill jig 180 may include a pair of hollow drill guides 182 operably coupled to a base 186. The guides 182 may correspond to the location of the staple legs 172 discussed previously such that the staple 170 used with the wedge 150 (e.g., the wedge 150c as shown in FIG. 7C) locates correctly into the holes 136 in the proximal phalanx 122. The practitioner may place the jig 180 with the guides 182 in the appropriate position over the proximal phalanx 122 using a handle 184 and drill into the proximal and distal portion 124, 126 of the proximal phalanx 122 through the hollow guides 182 using an appropriate drill bit.

In some embodiments, the material of the wedge 150 may be any material that is suitable for use inside the body of a patient and strong enough to stabilize the connection of the proximal and distal portions 124, 126 of the proximal phalanx 122. In the exemplary, non-limiting embodiments shown, the material is a polyether ether ketone (PEEK). PEEK provides the strength necessary for stability and prevents over-stiffening in the area of the wedge 150 because PEEK has substantially the same Young's modulus as the surrounding bone itself. Further, PEEK material is transparent on an X-Ray and therefore provides a better picture for the practitioner to assess how the bone is healing. Alternatively, any other known material having the same or similar characteristics as PEEK can be used.

In still other embodiments, more traditional securing methods may be used. For instance the use of a traditional staple, sutures, or any other securing method of the wedge to the surrounding bone structure may be used without deviating from the scope of the disclosure.

It should also be noted that the wedge embodiments and the related methods of inserting such wedge embodiments as disclosed or contemplated herein can also be used on other bones in the human body to address other, similar bone deformities.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A system for realigning a toe comprising:
(a) a saw blade configured to cut a single cut into a proximal phalanx of a toe, thereby creating proximal and distal portions of the proximal phalanx connected by a hinge portion;
(b) a wedge having:
(i) a perimeter having lateral and medial ends; and
(ii) an interior portion;
wherein the wedge has a lateral thickness and a medial thickness, and wherein the lateral thickness is larger than the medial thickness; and
(c) a connection member configured to secure the wedge to the proximal phalanx.

2. The system of claim 1, wherein the saw blade is configured to cut the single cut in a wedge shape into the proximal phalanx of the toe.

3. The system of claim 1, wherein the single cut has a lateral cut thickness, and wherein the lateral thickness of the wedge is greater than the lateral cut thickness of the single cut.

4. The system of claim 3, wherein the lateral thickness of the wedge is greater than the lateral cut thickness of the wedge-shaped cut by an amount that corresponds to a predetermined angle change thereby straightening the toe.

5. The system of claim 1, wherein the wedge comprises a polyether ether ketone (PEEK) material.

6. The system of claim 1, wherein the connection member comprises a suture.

7. The system of claim 1, wherein the connection member comprises a staple.

8. The system of claim 7, wherein the staple is integrally formed with the wedge.

9. The system of claim 8, wherein the staple comprises legs configured to couple to the proximal phalanx by locating the legs within staple holes drilled into each of the proximal and distal portions of the proximal phalanx.

10. A method of realigning a toe comprising:
(a) creating a hinge by cutting a single cut into the lateral side of a proximal phalanx of the toe;
(b) straightening the toe by rotating a distal portion of the proximal phalanx about the hinge, thereby creating a wedge opening defined by a space between the distal portion of the proximal phalanx, a proximal portion of the proximal phalanx, and the hinge;
(c) inserting a wedge into the wedge opening thereby providing support to the proximal phalanx; and
(d) securing the wedge to the proximal phalanx.

11. The method of claim 10, wherein the securing the wedge step is performed by suturing the wedge to the proximal phalanx.

12. The method of claim 10, wherein the securing the wedge step is performed by stapling the wedge to the proximal phalanx.

13. The method of claim 12, wherein the stapling the wedge step is performed by using a staple attached to the wedge, the staple having legs configured to couple to the proximal phalanx by locating the legs within staple holes drilled into each of the proximal and distal portions of the proximal phalanx.

14. The method of claim 13, wherein the staple is integrally formed with the wedge.

15. The method of claim 10, wherein the creating a hinge step is performed using a reciprocating saw.

16. The method of claim 15, wherein the reciprocating saw has a reciprocating saw blade.

17. The method of claim 16, wherein the reciprocating saw blade is configured to take a wedge shaped cut into the proximal phalanx of the toe.

18. A system for realigning a toe comprising:
(a) a wedge having:
(i) a perimeter;
(ii) lateral and medial ends; and
(ii) an interior portion;
wherein the wedge has a lateral thickness associated with the lateral end and a medial thickness associated with the medial end, and wherein the lateral thickness is larger than the medial thickness; and
(b) a connection member configured to secure the wedge to a proximal phalanx of the toe, wherein the proximal phalanx comprises:
(i) a proximal portion;
(ii) a distal portion separated from the proximal portion by a cut substantially through the proximal phalanx; and
(iii) a hinge portion connecting the distal portion to the proximal portion.

19. The system of claim 18, wherein the connection member is a staple formed integrally with the wedge, and wherein the staple comprises a pair of prongs that correspond to a pair holes in the proximal phalanx.

20. The system of claim 19, wherein one of the pair of holes is disposed in the proximal portion of the proximal phalanx, and the other of the pair of holes in disposed in the distal portion of the proximal phalanx.
